(19) United States
(12) Patent Application Publication
(10) Pub. No.: US 2012/0027824 A1
(43) Pub. Date: Feb. 2, 2012

(54) Preparations facilitated by vesicles using alkyl polypentosides and uses of said preparations

(76) Inventors: Cédric Ernenwein, Nouvion le Vineux (FR); Jean-François Tranchant, Marigny les Usages (FR); Thierry Pouget, Saint Cyr en Val (FR); Boris Estrine, Nanteuil la Foret (FR); Sinisa Marinkovic, Avancons (FR)

(21) Appl. No.: 13/066,678

(22) Filed: Apr. 21, 2011

(30) Foreign Application Priority Data
Apr. 23, 2010 (FR) FR 10 01755

(51) Int. Cl. A61K 8/14; A61K 47/26; B01J 13/18; A61K 8/60; A61L 9/012; C11D 17/08; A61K 9/127; A61K 8/06

(52) U.S. Cl. 424/401; 424/450; 514/777; 512/1; 510/418; 264/4.7

(57) ABSTRACT

Surfactant system capable of forming a dispersion of vesicles or liposomes constituted of alkyl polyglycoside of formula RO(X)n, in which R is a linear or branched alkyl chain, with or without unsaturation, and having 8 to 12 carbon atoms, X being a pentose residue, and n, representing the average degree of oligomerization, is between 1 and 3, and of a hydrotrope or co-solvent.

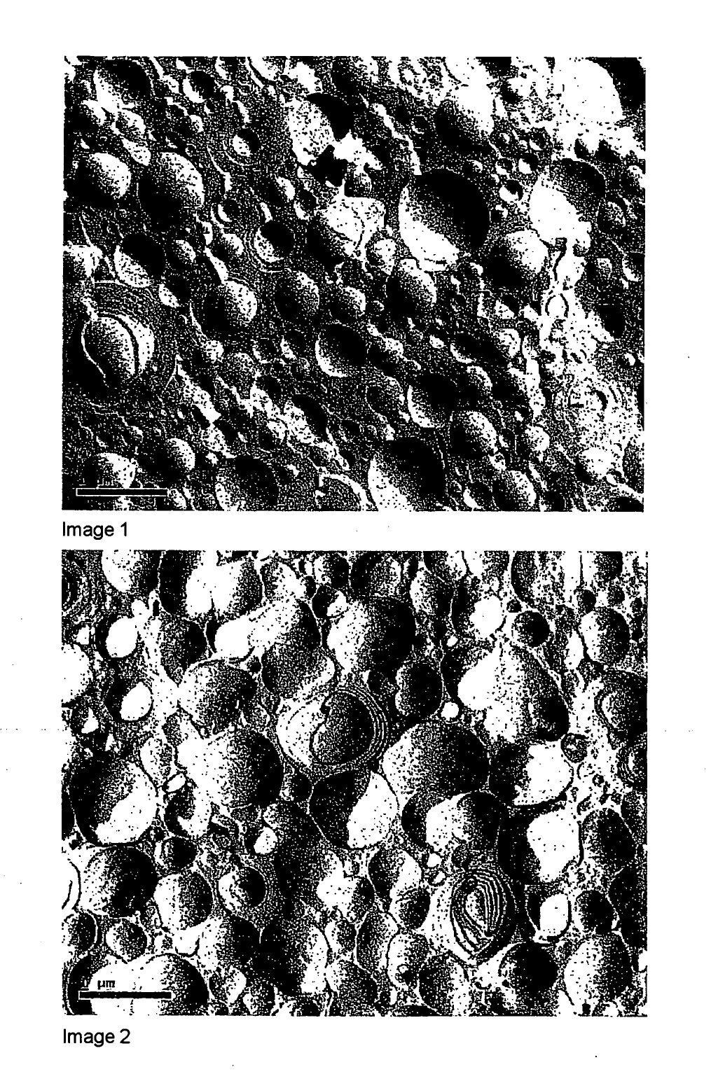

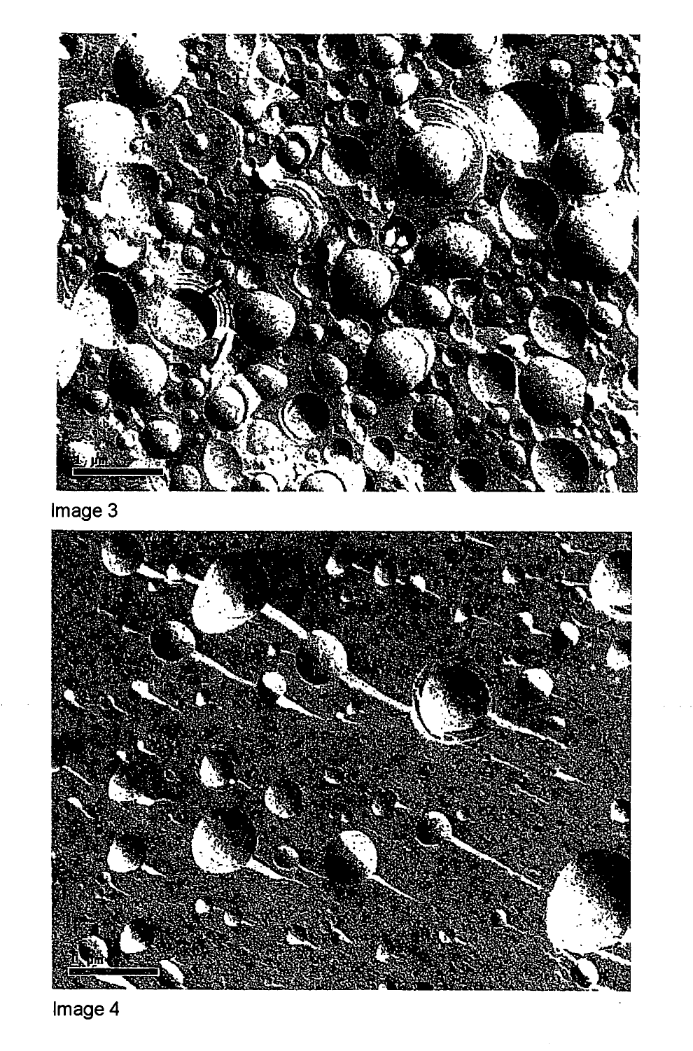

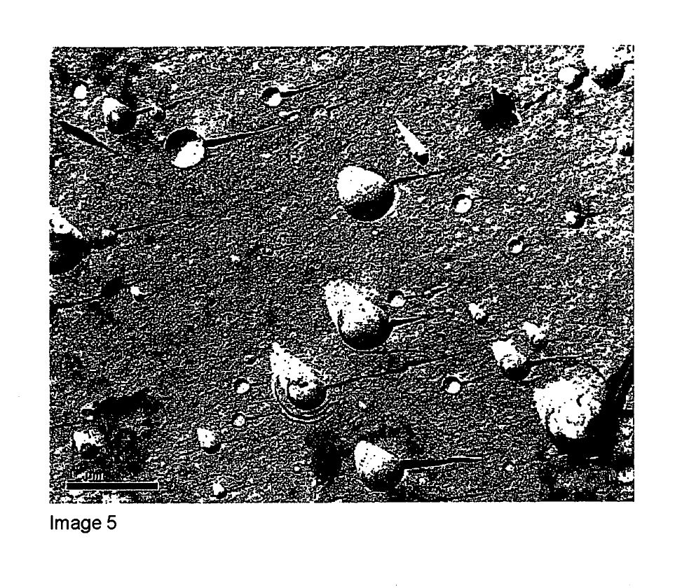

The present invention relates to a method for preparing a dispersion of vesicles or liposomes by means of at least one alkyl polypentoside. These organized systems can be used in various industries, such as cosmetics, pharmacy, agrochemistry, detergents, water treatment or decontamination of soil and aquifers.

Organized systems, vesicles or liposomes, are supramolecular aggregates of one or more layers of surfactants, forming a more or less spherical capsule of micron or sub-micron size (generally from 0.1 to 10 μm). These capsules can contain, in the layers, between the layers and within them, an aqueous or non-aqueous phase that can contain one or more dissolved or dispersed active substances, with the whole constituting the encapsulated phase or phases. This type of aggregation is also commonly called an onion phase, multilamellar vesicles or MLV when it is constituted of a stack of several layers of surfactants from the centre to the periphery and unilamellar vesicles or ULV or liposome when it is constituted of one or a few layers only and has a central cavity serving as a preferential place for encapsulation. There are a great many methods for preparing organized systems, vesicles or liposomes, notably described by R. G. Laughlin in Colloids Surfaces A: Physicochem. Eng. Aspects 128 (1997). The first method consists of applying high shear to a solution of surfactants or of lipids (generally in the form of a lamellar liquid crystal phase). This shear can be imposed by sonication or by forcing the liquid through calibrated orifices, or also by membrane filtration or by means of a high-pressure homogenizer. Another method consists of precipitating the vesicles by diluting a solution of surfactant in a suitable solvent or by dialysis. This method can be combined with shearing, to control the granulometric distribution and average size of the vesicles. The third procedure consists of hydrating a solid, generally lipid, film formed after evaporation of a solvent. Other methods employ injections of solvents in solutions or deposition of droplets of insoluble surfactants on polar surfaces and immersion in water. Finally, the last usual methods are chemical reactions, such as precipitation of fatty acids by acidification of the medium or polymerization reactions.

WO 93/19735 describes a method for preparing capsules of controlled size in relation to the shear imposed by a cell of the Couette type, starting from a concentrated solution of lamellar surfactants. WO 01/32146 describes capsules composed of amphiphilic copolymers stabilized by polymerization of end groups, for example acrylates. A method for preparing liposomes by concentration, drying, rehydration of the solid film, and then filtration is notably described in document US 2004057988A1. EP 023126A1 gives a method for preparing liposomes by the action of rapid cooling, generally under liquid nitrogen, of a lipid dispersion. Documents U.S. Pat. No. 4,752,425 and U.S. Pat. No. 4,737,323 give methods for preparing liposomes by injection of a lipid solution diluted in a solvent in an aqueous phase, then continuous removal of the solvent.

S. Segota in Advances in Colloid and Interface Science 121 (2006) describes structures of surfactants that are particularly suitable for forming vesicles and liposomes. The principal class of molecule possesses two or more lipophilic chains per polar head. These are mainly phospholipids and glycolipids, which can be extracted naturally or synthetically. There are also descriptions of vesicles using surfactants with double chains such as dialkyl-dimethyl-ammonium hydroxides or pairs of oppositely-charged surfactants generally called catanionic such as mixtures of sodium dodecyl benzene sulphonate (anionic) and didodecyldimethyl ammonium bromide (cationic).

Vesicles have also been observed with single-chain surfactants but generally in complex surfactant/co-surfactant/water mixtures, for example mixtures of ethoxylated fatty alcohols (such as lauric alcohol with 5 ethylene oxide units or C12E5) with incorporation of cholesterol as co-surfactant that is absolutely necessary for the formation of vesicles.

The alkyl polyglycosides constitute a class of non-ionic surfactants that are particularly appreciated on account of the renewable origin of the raw materials of which they are constituted, their relative mildness for the skin and mucous membranes, their ease of biodegradation and their low environmental impact.

DE 19634374A1 describes dispersions of vesicles formed with alkyl polyglucosides or APGs of formula:

RO(G)n (1)

in which R is a linear aliphatic radical having from 12 to 22 carbon atoms and n represents the degree of polymerization of the sugars or Dp, and is between 1 and 2 (product A). It is essential for the APG to be mixed with a linear fatty alcohol with 12 to 22 carbon atoms (product B) in a weight ratio A/B of 1/ (0.1 to 2). In other words, the C12 to C22 alcohol represents from 9.1 to 66.7 wt. % of the total weight of the system A+B. Preferably, the mixtures also contain lipid co-surfactants, such as sterols such as phytosterols, or dicetylphosphates, which are themselves known for forming liposomes readily.

D. Balzer in Nonionic Surfactants, Alkyl Polyglucosides, Surfactant Science Series Vol. 91, M. Dekker, N.Y., 2000 investigated the crystalline and liquid-crystal structures of APGs in solution. APGs possessing alkyl chains with 8 to 10 carbon atoms form lamellar phases in a range of concentration by weight from 78 to 81% (C8/C10 APG n=1.5). Longer APGs, having alkyl chains with 12 to 14 carbon atoms, form lamellar phases at a concentration at least greater than 65 wt. % (C12/C14 APG n=1.3).

Assuming that the formation of a lamellar phase constitutes a necessary condition for the formation of vesicles or liposomes, the APGs of formula (1) will therefore only be able to form such objects at concentrations at least greater than 65%.

Finally, U.S. Pat. No. 6,251,425 B from 2001 clearly demonstrates that APGs are primary surfactants that cannot form vesicles in the absence of steroid.

The complexity of manufacture and of application of vesicles and liposomes on the one hand, and the difficulty in easily obtaining said organized systems, that are stable and dilutable, with APGs alone, constitute technical drawbacks that the present invention is intended to correct.

The vesicles or liposomes of the present invention are prepared with at least one alkyl polyglycoside of formula:

RO(X) n (2)

in which R is a linear or branched aliphatic radical, with or without unsaturation, having 8 to 10 carbon atoms. X is a xylose residue in its alpha or beta isomeric form, of series L or D and in its furanose or pyranose form, n represents the average degree of oligomerization and is generally between 1 and 3, preferably between 1 and 1.5.

It was found, and this constitutes the basis of the present invention, that the alkyl polyglycosides of formula (2) can easily form a dispersion of organized systems, stable and dilutable vesicles or liposomes, even with linear alkyl chains with lengths less than or equal to 12 carbon atoms. “Easily” obtain organized systems, vesicles or liposomes, means the absence of co-surfactants, notably of steroids forming by themselves such systems or fatty alcohols, the surprising ease of application without the use of special mixers, for example sonicators or mixers of the Couette type, without the need to go via a solid phase obtained by precipitation or freezing, nor via solvent injection or solvent evaporation. “Stable” means durability and maintenance of the average size of the vesicles or liposomes formed according to the method of the invention as a function of storage time and storage temperature, generally between 4° C. and 60° C.

“Dilutable” means the possibility of dispersing a concentrated solution of vesicles or liposomes in a suitable solvent, generally aqueous, with a dilution factor from 2 to 1000 times, and preferably from 5 to 100 times, while ensuring the durability and the size of the initial objects. One of the characteristics of the invention is the surprising stability of the organized systems, vesicles or liposomes that have formed, even in complex formulations, for example emulsions, washing or detergent preparations, crop-protection formulations or preparations for soil decontamination.

The invention therefore relates to a method for preparing a dispersion of multilamellar vesicles or liposomes comprising dispersing a surfactant system that is able to form vesicles or liposomes in an aqueous phase, a solvent or an oil, characterized in that said surfactant system contains from 5 to 95 wt. %, preferably from 30 to 90 wt. %, of at least one alkyl polyglycoside of formula RO(X)n, in which R is a linear or branched alkyl chain, with or without unsaturation, and having 8 to 10 carbon atoms, X is the xylose residue, and n represents the average degree of oligomerization and is between 1 and 3.

The invention also relates to a method for enclosing one or more cosmetic, pharmaceutical, or phytopharmaceutical active substances, perfumes, enzymes, nutrients, trace elements, biological materials, essential oils, agents that are able to oxidize or degrade organic molecules, in multilamellar vesicles or liposomes, consisting of mixing said active substances, raw or diluted either in an oil, a solvent or in water with a surfactant system and then dispersing said mixture in an aqueous phase, a solvent or an oil, characterized in that said surfactant system contains from 5 to 90 wt. %, preferably from 30 to 90 wt. %, of at least one alkyl polyglycoside of formula RO(X)n, in which R is a linear or branched alkyl chain, with or without unsaturation, and having 8 to 10 carbon atoms, X is the xylose residue, and n represents the average degree of oligomerization and is between 1 and 3.

The term “dispersion” comprises either the dissolution, solubilization, formation of a suspension or of a colloidal solution in an aqueous or non-aqueous continuous phase.

The surfactant system according to the invention can be constituted by weight:
from 5 to 99%, preferably from 30 to 90%, of alkyl polyglycoside of formula RO(X)n, in which R is a linear or branched alkyl chain, with or without unsaturation, and having 8 to 10 carbon atoms, X being the xylose residue, and n, representing the average degree of oligomerization, is between 1 and 3, preferably between 1 and 1.5;
from 0.1 to 70% of one or more co-surfactants having an HLB at least equal to 10;
from 0 to 70%, preferably from 0.1 to 70%, of a hydrotrope or co-solvent selected from linear or branched C1 to C20 alcohols, glycol ethers, amyl, butyl, hexyl, 2-ethylhexyl, octyl, isononyl, isodecyl, octyldecyl glycosides, glycerol and partial derivatives of glycerol, terpinols, esters of organic acids such as acetic, formic, lactic, succinic, tartaric, glutaric, glutamic, adipic, lauric, oleic acid; and
from 0.1 to 70% of water.

The system can contain from 95 to 5 wt. %, preferably from 70 to 10 wt. %, of another alkyl polyglycoside, preferably a pentoside, but also a hexoside.

In order to ensure better stability and better dispersion of the vesicles or liposomes, it is possible to use, advantageously, a surfactant system having one or more alkyl polyglycosides of formula (2) and one or more co-surfactants and/or one or more co-solvents. The co-surfactant(s) do not in themselves necessarily form organized systems or liposomes. The weight ratio of alkyl polyglycoside to co-surfactant and/or co-solvent will then be between 0.5 and 1000, preferably from 1 to 100. In other words, the co-surfactant or co-solvent will preferably represent from 1 to 50 wt. % relative to the total weight.

Preferably, to obtain a dispersion that is stable and dilutable in an aqueous medium, a co-surfactant will be used with an HLB above 10, more preferably an HLB above 15. HLB stands for hydrophilic-lipophilic balance, notably described in “Agents de surface et émulsion” in the Galenica collection published by “Technique et Documentation (Lavoisier)” of 1983 and can be found by calculation using the Griffin method (Griffin WC: “Calculation of HLB Values of Non-Ionic Surfactants”, Journal of the Society of Cosmetic Chemists 5 (1954): 259) or the Davies method (Davies JT: “A quantitative kinetic theory of emulsion type, I. Physical chemistry of the emulsifying agent,” Gas/Liquid and Liquid/Liquid Interface. Proceedings of the International Congress of Surface Activity (1957): 426-438).

As co-surfactant it is possible to use, as examples but without intending to be limited to these: octyl, hexyl, 2-ethylhexyl, butyl, 2-methylbutyl or 3-methylbutyl polyglycosides, polyethoxylated sorbitan esters such as ethoxylated sorbitan esters of lauric, oleic, stearic, tri-oleic acid, polyethoxylated oils such as hydrogenated castor oil with 40 mol of ethylene oxide, highly ethoxylated fatty alcohols such as lauric alcohol with 23 ethoxylate units, oleic alcohol with 28 ethoxylate units. As co-solvents, it is also possible to use linear alcohols such as ethanol, butanol, pentanol, hexanol, octanol, decanol, dodecanol or branched alcohols such as 2-ethylhexanol, isoamyl alcohol, 3-methylbutanol, 2-butyloctanol, 2-butyldecanol, 2-hexyloctanol, 2-hexyldecanol, 2-octyldecanol, 2-hexyldodecanol, 2-octyldodecanol, 2-decyltetradecanol, terpinols as well as glycerol or its derivatives including monoglycerol esters, polyglycerols and its derivatives such as polyglycerol monostearate or polyglycerol polyricinoleate, butylene glycol, propylene glycol, glycol ethers such as diethylene glycol butyl-ether, diethylene glycol propyl-ether, triethylene glycol ethyl-ether, triethylene glycol methyl-ether, triethylene glycol butyl-ether and their acetates, it is also possible to use esters of fatty acids such as methyl, butyl, isobutyl, amyl, propyl, isopropyl, 2-ethylhexyl, hexyl, octyl, decyl, isodecyl esters of C12 to C24 fatty acids, saturated or having one or more unsaturations.

Preferably, to obtain a dispersion that is stable and dilutable in a non-aqueous medium, it is possible to use, advantageously, a co-surfactant with HLB below 10 or preferably below 7, more preferably below 5, instead of a co-surfactant with HLB at least equal to 10. As examples and without intending to be limited to these, the following can be selected as co-surfactant of low HLB: sorbitan esters such as sorbitan monolaurate, monooleate, monostearate, trioleate, tristearate, mildly ethoxylated sorbitan esters such as POE(2) sorbitan stearate, propylene glycol esters of fatty acids such as propylene glycol monooleate, propylene glycol monomyristate, glycerol monoesters such as glycerol monooleate, glycerol monostearate, sugar esters such as sucrose myristate, palmitate or stearate, sucrose di-, tri- and polystearate, sucrose di-, tri- and polypalmitate, sucrose di-, tri- and polylaurate, mildly ethoxylated fatty alcohols such as cetyl alcohol POE(2), oleic alcohol POE(2), mildly ethoxylated oils such as hydrogenated castor oil with 5 moles of ethylene oxide.

Images 1-5 are images of cryofracture replicas of solutions of the surfactant systems of the invention with a transmission electron microscope.

The invention finds application in many fields. It relates to vesicles or liposomes that contain from 1 to 99 wt % relative to the total weight of the vesicles or liposomes, preferably from 10 to 95% and more preferably from 30 to 90%, of one or more cosmetic, pharmaceutical, or phytopharmaceutical active substances, perfumes, enzymes, nutrients, trace elements, biological materials, essential oils, agents capable of oxidizing or degrading organic molecules, raw or diluted, whether in an oil, a solvent or in water. The invention also relates to an oil-in-water emulsion, aqueous or aqueous-alcoholic solution, aqueous or aqueous-alcoholic gel containing from 0.01 to 90 wt %, preferably from 0.1 to 50% and more preferably from 1 to 20 wt % of vesicles or liposomes and prepared using at least one surfactant system according to the invention, as well as a water-in-oil emulsion, solvents or mixtures of solvents, microemulsion, containing from 0.01 to 90 wt %, preferably from 0.1 to 50 wt % and more preferably from 1 to 10 wt % of vesicles or liposomes and prepared using at least one surfactant system according to the invention.

### Cosmetics and (Dermo) Pharmacy

The invention will be used in the field of cosmetics, dermo-pharmacy and pharmacy for encapsulating active substances notably for releasing them over time or as a function of external stresses such as temperature rise, application of shearing or chemical reactions such as changes in pH, reactions of hydrolysis or oxidation, or enzymatic reactions. It is also possible to try to encapsulate substances that are sensitive, notably to oxidation or to exposure to UV radiation, with the aim of protecting them and maintaining their efficacy. It is also possible to try to transport vesicles to targeted action sites through the dermis, epidermis or hypodermis or absorb them notably on the skin or the hair in order to prolong the desired effect.

A particular feature of the invention is the production of vesicles that are stable even in complex environments, notably in emulsions and concentrated solutions of surfactants.

The vesicles prepared by the methods of the invention will therefore be used in particular in aqueous preparations in the form of solutions, dispersions, gels, lotions and in oil-in-water (O/W) emulsions, water-in-oil (W/O) emulsions or complex systems such as multiple emulsions (W/O/W or O/W/O), notably in preparations such as creams, milks, ointments, unguents, gels, lotions.

As the vesicles prepared according to the methods of the present application can be dispersed both in aqueous and non-aqueous media, they can also find application in areas containing little or no water. We may mention for example the formulation of massage oil, sunscreen oils, oils for sportsmen or for treatment of the skin. The invention will also find application in solid or pasty preparations such as in make-up, such as lipsticks, mascaras or foundations.

The encapsulated substances can be for example vitamins, such as vitamin A, E or C, synthetic or natural perfumes such as benzyl acetate, linalyl acetate, linalyl benzoate, citral, citronellal, lilial, eugenol, citronellol, linalool, terpene derivatives, essences of sage, of chamomile, of carnation, of vetiver, of hybrid lavender, essential oils such as essential oils of lavender, thyme, savory, sage, mint, cumin, caraway, green anise, fennel, dill, eucalyptus, cajeput, niaouli, clove, pine, cedar, cypress, juniper, lemon, orange, bergamot, cinnamon, bay, chamomile, anti-inflammatories such as plant extracts, alpha-bisabolol, panthenol, alpha-tocopherol, agents against burns such as allantoin, anti-ageing agents such as retinol, self-tanning agents such as dihydroxyacetone (DHA), depigmenting agents such as kojic acid, coumaric acid, arbutin, slimming agents such as caffeine, antiperspirants such as salts of aluminium, of zinc or of zirconium, diethylene triamine pentaacetic acid, anti-dandruff agents such as pyrithione of zinc or of aluminium, salicylic acid, pyridone salts and piperazine derivatives, UV filters such as benzophenone derivatives, esters of cinnamic acids, esters of salicylic acid, 3-benzylidene camphor, antioxidants such as ascorbic acid and derivatives thereof, citric acid and derivatives thereof, glutamic acid, glutamates and derivatives thereof, lactic acid and derivatives thereof, tartaric acid and derivatives thereof, bioflavonoids, butylhydroxy-hydroxyanisole, carotene and derivatives thereof, sulphites such as bisulphites of sodium, chlorobutanol, preservatives such as parabens, phenoxyethanol, 2-bromo-2-nitropropane-1,3-diol, formaldehydes, pantanediol, sorbic acid, hydrating agents such as glycerol, sorbitol, collagen, pro-collagen, gelatin, aloe vera, hyaluronic acid, urea, insect repellents such as acetamiprid, etofenprox, permethrin, cypermethrin, N,N-diethyl-m-toluamide, butylacetylaminopropionate, pharmaceutical active ingredients, such as disinfectants such as chlorhexidine gluconate, benzalkonium chloride, benzoic acid, cetylpyridinium chloride, anti-inflammatories such as arnica tincture, eucalyptol, menthol, dimethoxy-1,2-benzene, anti-acne agents such as tretinoid derivatives, azelaic acid, salicylic acid, antifungals such as derivatives of pyridone such as cyclopiroxolamine, derivatives of imidazole such as clotrimazole, folic acid, riboflavin, sequestering agents such as mucic acid, phytic acid, NTA, EDTA, colorants, pH adjusters, natural or synthetic aromas.

### Detergency

The organized systems, vesicles or liposomes prepared according to the methods of the present invention will be used in detergent formulations notably in detergents, emollients, products for cleaning hard surfaces, dishwashing products or products for cleaning vehicles.

In the area of detergents, notably liquid detergents or gels, the encapsulated substances can be, for example, perfumes notably for prolonging the “fresh” effect and pleasant odour of clean linen or for protecting them from the enzymes present in the formulation, notably lipases which can denature the latter during the washing cycle; the enzymes can also be encapsulated in order to protect them from UV radiation, and thermal or chemical degradation notably due to alkaline pH. The vesicles can therefore also contain UV absorbers, bleaching agents or optical brighteners, as well as bleaching activators, non-ionic or cationic vitamins, anti-microbial, anti-fungal or anti-viral agents, antiperspirants, deodorants, nutrients, agents for protecting the skin and skin hydrating agents.

As perfumes and fragrances, we may mention for example, and without intending to be limited to these, synthetic or natural perfumes such as benzyl acetate, linalyl acetate, linalyl benzoate, citral, citronellal, lilial, eugenol, citronellol, linalool, terpene derivatives, essences of sage, of chamomile, of carnation, of vetiver, of hybrid lavender, essential oils such as essential oils of lavender, thyme, savory, sage, mint, cumin, caraway, green anise, fennel, dill, eucalyptus, cajeput, niaouli, clove, pine, cedar, cypress, juniper, lemon, orange, bergamot, cinnamon, bay, chamomile.

As bleaching agents, it is possible to use for example hydrogen peroxide, sodium hypochlorite, percarbonates of potassium, perborates of sodium or peracids.

As bleaching activators, we may mention for example N,N,N′,N′-tetraacetyl ethylenediamine or derivatives thereof, sodium nonanoyloxybenzene sulphonate and derivatives thereof or sodium 4-benzoyloxybenzene sulphonate.

The enzymes make it possible to complete the washing action of the preparations, notably on soiling with fats and foodstuffs, and it is possible for example to use amylases, cellulases, lipases, peroxidases, and proteases marketed notably by the company Novosyme.

### Crop Protection Formulation

The invention also finds application in the area of agrochemicals notably for the formulation of herbicides, insecticides, fungicides, algicides, rodenticides, molluscicides, acaricides, growth regulators, nutrients, trace elements, fertilizers, elicitors, repellents against insects, mammals or birds, baits for insects or rodents. The crop protection formulations can be in the form of aqueous or non-aqueous solutions, oil-in-water (O/W) emulsions or water-in-oil (W/O) emulsions, microemulsions, suspo-emulsions, gels, emulsifiable concentrates, granules, wettable powders, tablets or sticks that can be broken up or dispersed.

The objective of encapsulating a substance in vesicles of the invention is to permit accurate dosage and controlled release of the active ingredients, to reduce the toxicity of pesticides or to protect biologically active substances notably against external stresses such as UV radiation, chemical reactions such as those induced by changes in pH or complexation with ions notably of calcium or of magnesium present in hard water.

As pesticides that can be encapsulated by the vesicles of the invention, we may mention for example, and without intending to be limited to these, herbicides of the amide type such as benzipram, cyprazole, fomesafen, anilides such as metamifop, pentanochlor, arylalanines, chloroacetanilides such as alchlor, metazochlor, propachlor, terbuchlor, sulphonanilides such as benzofluor, pyrimisulfan, benzoic acids such as dicamba, 2,3,6 TBA, tricamba, phthalic acids, picolinic acids, quinoline-carboxylic acids, benzoylcyclohexanediones, benzofuranyl-alkylsulphonates, carbamates, carbanilates such as chlorobufam, chloropropham, desmedipham, phenmedipham, phenmedipham-ethyl, cyclohexene oximes such as cloproxydim, cycloxydim, profoxydim, cycloproxylisoxazoles, dicarboximidines such as benzfendizone, flumixazin, dinitroanilines, dinitrophenols, diphenyl-ethers such as ethoxyfen, dithiocarbamates, halogenated aliphatic herbicides, imidazolinones, nitriles such as bromoxynil, chloroxynil, dichlobenil, organophosphate herbicides such as 2,4 DEP, fosamine, glufosinate, glyphosate, oxadiazolonz, phenoxy herbicides such as 2,4 DEB, etnipromid, 2,4D, MCPA, 2,4 DB, dichloprop, mecoprop, diclofop, fenaxoprop, fluazifop, clodinafop, haloxifiop, phenylenediamines, pyrazoles such as metazochlor, benzofenap, pyridazines, pyridazinones, pyridines such as aminopyralid, pyrimidinediamines, thiocarbamates such as sulfallate, thiobencarb, thiocarbonates, triazines such as trihydroxytriazine, atrazine, cyprazine, atraton, prometon, aziprotryne, triazinones, triazoles, triazolones such as bencarbazone, propoxycarbazone, triazolopyrimidines, herbicides based on urea such as phenylureas, sulphonylureas such as amidosulfuron, ethoxysulfuron, mezosulfuron, chlorosulfuron, metsulfuron, triasulfuron, thiadiazolylureas such as buthiron, thiazafluron as well as quaternary ammoniums and derivatives thereof, inorganic herbicides such as copper sulphate, ammonium sulphamate, sodium chlbrate, potassium cyanate.

As insecticides that can be encapsulated we may mention for example those derived from plants for example d-limonene, nicotine, pyrethrins, jasmolins, pyrethroids, carbamate insecticides such as aryl-methylcarbamates, heterocyclic monomethyl or dimethylcarbamates such as carbofuran, carbosulfan, primicarb, carbamate oximes such as oxamyl, thiofanox, methomyl, organochlorine insecticides such as DDT, methoxychlor, hexachlorocyclohexane (HCH) and derivatives thereof, heptachlor, mirex, organophosphate insecticides such as orthophosphates (chlorfenvinphos, dichlorvos), phosphorothionates (bromphos, diazinon, parathion), phosphorothiolates, phosphorothiolothionates (dimethoate, disulfoton, menazon), phosphonates (triclorphon, butonate), pyrophosphoramides (shradan), insecticides blocking insect growth such as chitin inhibitors such as novaluron, penfluron, natural or synthetic juvenile hormones such as methoprene, kinoprene, formamidine insecticides such as chlorodimeform, amitraz, insecticides derived from benzoylphenylurea such as diflubenzuron, penfluron, inorganic insecticides such as borax, copper oleate, sodium thiocyanate.

As fungicides that can be encapsulated according to the invention we may mention for example copper fungicides, such as Bordeaux mixture, copper oxychloride, copper oxides, copper hydroxides, fungicides based on mercury such as organomercury agents such as PMA, methoxyethylmercury acetate, thiomersal, dithiocarbamates such as thiram, maneb, phthalimides such as captan, phthalonitriles such as chlorothalonil, dicarboximides such as vinclozolin, dinitrophenols such as dinocap, dinocton, benzimidazoles such as benomyl, cypendazole, oxathiines such as carboxin, morpholines such as tridemorph, carbamorph, hydroxyaminopyrimidines such as ethirimol, antibiotic fungicides such as kazugamycin, strobilurins and derivatives thereof, phenylamides as metalaxyl, organophosphorus compounds such as pyrazophos, fosetyl, imidazoles such as cyazofamid, irpodione, prochloraz, triazoles such as hexaconazole, epoxiconazole, derivatives of guanidine, chlorinated aromatic compounds such as quintozene, dichloran, chlorothalonil, dicloran.

### Application for Water Treatment and Decontamination of Soil and Aquifers

The invention also finds application in the area of the decontamination of soil and aquifers and the treatment of water, notably in septic tanks.

It is already known that incorporation of an agent permitting oxygenation of the environment made it possible to increase the performance of operations of bioremediation of contaminated soils, notably by hydrocarbons. The objective is to increase the number of bacteria capable of biodegrading the pollutants present by injecting formulations that are able, generally by chemical reactions, to supply oxygen to the environment. This type of preparation is notably described in document U.S. Pat. No. 5,264,018.

In the case of pollution of aquifers and groundwater, notably by chlorinated pollutants, it may be advantageous to incorporate chemicals that initiate reactions of oxidation, either for degrading the pollutant completely and directly, or for making the pollutant more easily accessible by microorganisms for its complete biodegradation. These types of compounds are for example Fenton's reagents, such as those described in document U.S. Pat. No. 6,268,205. However, in all cases, the reagents may be relatively unstable, they can degrade rapidly and lose much of their efficacy before they reach their site of action. It is therefore advantageous to encapsulate, in vesicles prepared according to the invention, reagents permitting oxygen and/or hydrogen to be released and/or the pollutants to be oxidized. This is in order to guarantee their transport to the zone to be treated, to permit controlled release for maintaining long-term efficacy or to reduce the toxicity of said substances for the environment.

As examples and without intending to be limited to these, the substances that can be encapsulated by a dispersion of vesicles of the invention will be hydrogen peroxide, urea/hydrogen peroxide complexes, sodium percarbonate, calcium peroxide, magnesium peroxide, potassium permanganates, sodium persulphate, sodium bisulphite. Catalysts, nutrients rich in trace elements, enzymes, bacteria, pH adjusters, and complexing agents can also be encapsulated in order to potentiate the action of oxygenation, bioremediation or oxidation of the medium being treated.

### Preparation

Preferably the substances to be encapsulated are liquid or are dissolved in a liquid. They can therefore be diluted in water, a solvent or an oil to a concentration permitting their complete dissolution. The active substances and optionally their solvent thus constitute the phase to be encapsulated.

The phase to be encapsulated represents from 1 to 99 wt % relative to the total weight of vesicles or liposomes, preferably from 10 to 95% and more preferably from 30 to 90%.

The vesicles or liposomes will be prepared by intimately mixing the phase to be encapsulated with the surfactant system according to the invention at room temperature or at a higher temperature but preferably below 100° C. Mixers with low shear will preferably be selected, such as pendulum-type oscillators equipped with one or more propellers or an anchor, double-rotation stirrers equipped with central stirring with one or more propellers and peripheral stirring equipped with one or more scraping blades matching the shape of the reactor. It is also possible to use kneaders and mixers. It will be preferable to avoid stirrers with high shear such as rotor-stators, high-speed serrated propellers, horizontal stirrers equipped with a shaft and blades rotated by a motor, colloid mills. The duration of stirring can vary from a few minutes to several hours, until a viscous liquid is obtained, generally having a viscosity measured by a Brookfield viscosimeter from 100 to 500 000 centipoise at the temperature of the mixture or of a homogeneous paste (notably at 25° C.). This concentrated preparation of vesicles or liposomes can be used immediately for the preparation of pharmaceutical products, cosmetics, detergents, for the treatment of water or of aquifers or can be stored at a temperature of use, generally from 4 to 45° C. or higher but preferably below 100° C. In order to increase the stability of the concentrate, it may be necessary to add agents for blocking or slowing bacterial or fungal proliferation. In this case they will be added during the mixing phase and at concentrations guaranteeing their efficacy.

In cosmetic preparations constituted of an aqueous continuous phase, i.e. in the form of solution, gel or oil-in-water emulsion, the proportion of vesicles or liposomes will vary from 0.01 to 50% relative to the total weight of the preparation, preferably from 0.1 to 20%. In the case of inverted preparations, notably water-in-oil emulsions, the rate of incorporation will generally be from 0.01 to 30% relative to the total weight and preferably from 0.1 to 10%.

In detergent preparations, notably those for encapsulating one or more enzymes in liquid detergents or gels, the vesicles or liposomes can contain from 2 to 15% of enzymes relative to their weight and will be incorporated at a level from 0.1 to 15 wt % relative to the total weight of the preparation.

For the encapsulation of perfumes or of essential oils, for prolonging the fresh effect of linen for example, the rate of incorporation of vesicles or liposomes in the preparations will vary for example from 0.1 to 20% relative to the total weight of the preparation.

In the case of plant protection preparations, the concentration of active substances in the vesicles or liposomes and the rate of incorporation in the preparations will depend on the nature of said active substances and on the regulations of the country authorizing their use. The rate of incorporation can vary for example from 0.01 to 10 wt % of the preparation for very effective active molecules such as those in the sulphonyl-urea group such as metsulfuron-methyl, or from 10 to 90 wt % of the preparation for herbicides that are less active, for example 2,4D, dimethylamine salt.

For the treatment of water and septic tanks, application of a preparation containing from 0.1 to 90 wt % of vesicles or liposomes, preferably from 1 to 50%, and more preferably from 5 to 30% will be carried out discontinuously. For the treatment of aquifers and groundwater, treatments with a preparation containing from 0.01 to 50 wt % of vesicles or liposomes, preferably from 0.1 to 10% can be applied discontinuously or continuously throughout the operation of decontamination.

### Method of Characterization of Vesicles and Liposomes

The method used for characterization consists of observation of cryofracture replicas of solutions of surfactants with a transmission electron microscope. This method is widely documented, notably by A. GULIK, L. P. AGGERBECK, J. C. DEDIEU and T. GULIK-KRZYWICKI, in Journal of Microscopy, Vol. 125, Pt 2, February 1982, pp. 207-213, or more recently by 0. MONDAIN-MONVAL in Current Opinion in Colloid and Interface Science, 10 (2005), 250-255.

The method that was used is freeze etching, which produces a replica of the structure that can be observed by transmission electron microscopy. This technique has four main stages:
1 freezing;
2 fracture and “etching”;
3 shading and formation of the replica;
4 cleaning the replica.

Finally, images of the replica are obtained using a transmission electron microscope, followed by visual analysis of the images.

For compliance with the invention, the images must show a homogeneous dispersion of spherical or quasi-spherical objects in positive and negative and having, in the case of multilayered vesicles, several spherical streaks at the periphery reflecting the build-up of several layers. Moreover, the images must not show extensive cleavage zones, represented by linear streaks and reflecting the presence of a lamellar phase that has not wrapped round, resulting from a system that is unable to form vesicles or liposomes spontaneously.

The invention will be explained in more detail in the following examples, given solely for purposes of illustration and in which the characteristics of the products obtained are evaluated according to the criteria described above.

### EXAMPLE 1

### Demonstration of the Multilayered Vesicles According to the Invention

5.25 g of octyl/decyl poly-xylosides (XYLC8/10 DP=1.13) are mixed with 44.75 g of water/glycerol using a mechanical stirrer at 500 rev/min for 30 minutes at 80° C., then cooled to room temperature while maintaining stirring. A cryofracture replica is then made from this preparation and is observed in transmission electron microscopy (TEM).

The image obtained (Image 1) clearly shows the presence of numerous vesicles from 0.2 to 1 pm. Numerous circular grooves can be seen, reflecting the formation of multilayered vesicles.

### EXAMPLE 2

### Demonstration of the Robustness of the Multilayered Vesicles According to the Invention

A decyl poly-xyloside (Xyl 010) is dispersed in a water/glycerol solution at various concentrations using a mechanical stirrer at 500 rev/min for 30 minutes at 80° C., and then cooled to room temperature while maintaining stirring.

A sample is taken from each dispersion to obtain a cryofracture replica, which is then observed in the TEM. The persistence of the multilayered vesicles is verified by the presence of characteristic objects.

The results obtained are presented in the following table and in images 2 to 5.

| | Dilution factor | | | |
|---|---|---|---|---|
| | 1 | 2 | 8 | 16 |
| % XYLC10 | 15 | 7.5 | 1.875 | 0.9375 |
| DP = 1.5 | | | | |
| Observation | Homogeneous | Homogeneous | Single-layer | Single- |
| in TEM | dispersions | dispersions | structures | layer |
| | of | of | mixed with | structures |
| | multilayered | multilayered | some | mixed |
| | vesicles | vesicles | multilayered | with |
| | | | | some |
| | | | | multi- |
| | | | | layered |
| Image No. | 2 | 3 | 4 | 5 |

### COMPARATIVE EXAMPLE 1

Decyl poly-xylosides (XylC10 DP=1.25) and decyl poly-glucosides (GluC10 DP=1.3) are dispersed at 15 wt % in a water/glycerol mixture according to the protocol in example 1.

The proportion of GLUC10 Dp1.3 in the surfactant mixture is designated R. This proportion varies from R=1 for a solution containing only Glu C10 Dp=1.3 to R=0 for a solution containing only XYL C10 DP=1.25. A cryofracture replica is obtained for each of the dispersions and is then observed in the TEM. Observation of multilayered vesicles is indicated by MLV, observation of a lamellar phase that has not wrapped round is indicated by L and observation of a micellar phase is indicated by MC.

| R | 1 | 0.9 | 0.8 | 0.7 | 0.6 | 0.5 | 0.4 | 0.3 | 0.2 | 0.1 | 0 |
|---|---|---|---|---|---|---|---|---|---|---|---|
| Observations | MC | MC | MC | MC | MC | MC | MC | L | MLV | MLV | MLV |
| in TEM | | | | | | | | | | | |

This example shows that it will be necessary to have at least 70 wt % of alkyl pentosides (xylC10 DP=1.25) in the surfactant mixture to obtain a lamellar phase at the test dilution and at least 80 wt % to obtain a homogeneous dispersion of multilayered vesicles.

Decyl polyglucoside and the mixtures containing from 40 to 90% of this polyglucoside do not form vesicles, thus demonstrating the specificity of the alkyl poly-xylosides for forming said objects.

### EXAMPLE 3

### Aqueous Dispersion of Vesicles Stable at Low Temperature According to the Invention

Mixtures of decyl poly-xyloside (Xyl C10 DP=1.5) and of co-surfactants with different HLB values are dispersed at 7.5 wt % in a water/glycerol mixture according to the protocol described in example 1. The ratio of surfactant to co-surfactant is equal to 10. For each of the co-surfactants used, the dispersions obtained are stored at 5° C. for 3 months and are then observed visually and with a light microscope (magnification x10). Cryofracture replicas are obtained and observed in the TEM for all of the samples before the investigation of stability at 5° C. and then on the samples that are stable after 90 days at 5° C.

The following table summarizes the results obtained in relation to the HLB (Hydrophilic/Lipophilic Balance) of the co-surfactant.

| HLB of co- | Presence of MLV | Presence of MLV |
|---|---|---|
| surfactant | (t = 0) | (t = 90 d at 5° C.) |
| 4.7 | Yes | — |
| 10.6 | Yes | — |
| 14.2 | Yes | Yes |
| 16.7 | Yes | Yes |

The presence of co-surfactants of high HLB (>14) gives good stability and a homogeneous dispersion of multilayered vesicles even after 3 months at 5° C. The use of co-surfactants of low HLB (<11) shows deterioration of the multilayered vesicles in these conditions of storage.

### EXAMPLE 4

### Oily Dispersion of Vesicles According to the Invention

A first dispersion is prepared by mixing, using a mechanical stirrer at 500 rev/min and at 50° C., 45 g of octyl/decyl poly-xyloside (XYL C8/C10 DP=1.3) with 24 g of sorbitan oleate (Radia 7125 from the company OLEON, HLB=4.7) and 31 g of water. After switching off the heating, mechanical stirring of the mixture thus obtained is continued until it reaches the temperature of the laboratory.

45 g of this mixture are then dispersed in 55 g of paraffin oil (Marcol 82) with mechanical stirring and at the temperature of the laboratory.

This gives a homogeneous dispersion of liquid crystal phase in oil, and observation, with a light microscope, of a sample placed between two crossed polarizers shows the presence of features that are characteristic of vesicles.

### COMPARATIVE EXAMPLE 2

On the one hand, dodecyl polyxylosides (Xyl C12 DP=1.3) are dispersed at 3.8 wt. % in a water/glycerol mixture according to the procedure in example 1.

On the other hand, 2-butyloctyl polyxylosides (Xyl Iso12 DP=1.3) are dispersed at 15 wt. % in a water/glycerol mixture according to the procedure in example 1.

The first preparation gives a heterogeneous mixture with sedimentation of the surfactant system. Observation of the upper phase in the TEM does not show the presence of vesicles.

The second preparation gives a slightly turbid solution. Observation of the solution in the TEM shows the presence of a lamellar phase that is not coiled and shows the complete absence of vesicles.

This example shows that alkyl xylosides with degrees of polymerization between 1 and 1.5 t with long chains of more than 12 carbon atoms are not easily able to form vesicles according to the invention.

1. Method for preparing a dispersion of multilamellar vesicles or liposomes comprising dispersing a surfactant system that is able to form vesicles or liposomes in an aqueous phase, a solvent or an oil, characterized in that said surfactant system contains from 5 to 99 wt. %, of at least one alkyl polyglycoside of formula RO(X)n in which R is a linear or branched alkyl chain, with or without unsaturation, and having 8 to 10 carbon atoms, X is the xylose residue, and n represents the average degree of oligomerization and is between 1 and 3.

2. The method of claim 1, wherein the surfactant system contains from 70 to 90 wt. % of the at least one alkyl polyglycoside.

3. Method for enclosing one or more cosmetic, pharmaceutical, or phytopharmaceutical active substances, perfumes, enzymes, nutrients, trace elements, biological materials, essential oils, agents that are able to oxidize or degrade organic molecules, in multilamellar vesicles or liposomes, comprising mixing said active substances, raw or diluted in an oil, a solvent or in water with a surfactant system, then dispersing said mixture in an aqueous phase, a solvent or an oil, characterized in that said surfactant system contains by weight from 5 to 99 wt. %, of at least one alkyl polyglycoside of formula RO(X)n in which R is a linear or branched alkyl chain, with or without unsaturation, and having 8 to 10 carbon atoms, X is the xylose residue, and n represents the average degree of oligomerization and is between 1 and 3.

4. The method of claim 3, wherein the surfactant system contains from 70 to 90 wt. % of the at least one alkyl polyglycoside.

5. Surfactant system, which comprises by weight:
from 5 to 99%, of alkyl polyglycoside of formula RO(X)n, in which R is a linear or branched alkyl chain, with or without unsaturation, and having 8 to 10 carbon atoms, X being the xylose residue, and n, representing the average degree of oligomerization, is between 1 and 3, preferably between 1 and 1.5;
from 0.1 to 70% of one or more co-surfactants having an HLB at least equal to 10; and
from 0.1 to 70% of water.

6. The system of claim 5 which comprises from 30 to 90% of said alkyl polyglycoside.

7. The system of claim 5, which comprises by weight:
from 0 to 70% of a hydrotrope or co-solvent selected from linear or branched C1 to C20 alcohols, glycol ethers, amyl, butyl, hexyl, 2-ethylhexyl, octyl, isononyl, isodecyl, octyldecyl glycosides, glycerol and partial derivatives of glycerol, terpinols, esters of organic acids such as acetic, formic, lactic, succinic, tartaric, glutaric, glutamic, adipic, lauric, oleic acid.

8. Surfactant system which comprises by weight:
from 5 to 99%, of alkyl polyglycoside of formula RO(X)n, in which R is a linear or branched alkyl chain with or without unsaturation, and having 8 to 10 carbon atoms, X being the xylose residue, and n, representing the average degree of oligomerization, is between 1 and 3, preferably between 1 and 1.5;
from 0.1 to 70% of one or more co-surfactants having an HLB below 10; and
from 0.1 to 70% of water.

9. The system of claim 8 which comprises from 30 to 90% of said alkyl polyglycoside.

10. The system of claim 8, which comprises by weight:
from 0.1 to 70% of a hydrotrope or co-solvent selected from linear or branched C1 to C20 alcohols, glycol ethers, amyl, butyl, hexyl, 2-ethylhexyl, octyl, isononyl, isodecyl, octyldecyl glycosides, glycerol and partial derivatives of glycerol, terpinols, esters of organic acids such as acetic, formic, lactic, succinic, tartaric, glutaric, glutamic, adipic, lauric, oleic acid.

11. The system of claims 5, which is in the form of vesicles or liposomes that contain from 1 to 99 wt. % relative to the total weight of vesicles or liposomes, of one or more cosmetic, pharmaceutical, or phytopharmaceutical active substances, perfumes, enzymes, nutrients, trace elements, biological materials, essential oils, agents that are able to oxidize or degrade organic molecules, raw or diluted in an oil, a solvent or in water.

12. The system of claim 11, which is in the form of an oil-in-water emulsion, aqueous or aqueous-alcoholic solution, aqueous or aqueous-alcoholic gel that contains from 0.01 to 90 wt. % of vesicles or liposomes.

13. The system of claim 11, which is in the form of a water-in-oil emulsion, solvents or mixtures of solvents, microemulsion containing from 0.01 to 90 wt. % of vesicles or liposomes.

14. The systems of claim 12 which is in the form of a water-in-oil emulsion, solvents or mixtures of solvents, microemulsion containing from 1 to 20 wt. % of vesicles or liposomes.

15. The systems of claim 13 which is in the form of a water-in-oil emulsion, solvents or mixtures of solvents, microemulsion containing from 1 to 10 wt. % of vesicles or liposomes.

16. Method of formulation of cosmetic, pharmaceutical, phytopharmaceutical, or detergent products, for water treatment or soil decontamination, comprising using a dispersion of vesicles or liposomes in the form of an oil-in-water emulsion, aqueous or aqueous-alcoholic solution, aqueous or aqueous-alcoholic gel that contains from 0.01 to 90 wt. %, of vesicles or liposomes, said vesicles or liposomes comprising from 5 to 99 wt. % of at least one alkyl polyglycoside of formula RO(X)n in which R is a linear or branched alkyl chain, with or without unsaturation, and having 8 to 10 carbon atoms, X is the xylose residue, and n represents the average degree of oligomerization and is between 1 and 3.

17. The method of claim 16 which comprises dispersing vesicles or liposomes in the form of a water-in-oil emulsion, solvents or mixtures of solvents, microemulsion containing from 1 to 20 wt. % of vesicles or liposomes.

18. Method of formulation of cosmetic, pharmaceutical, phytopharmaceutical, or detergent products, for water treatment or soil decontamination, comprising dispersing vesicles or liposomes in the form of a water-in-oil emulsion, solvents or mixtures of solvents, microemulsion containing from 0.01 to 90 wt. %, of vesicles or liposomes, said vesicles or liposomes comprising from 5 to 99 wt. % of at least one alkyl polyglycoside of formula RO(X)n in which R is a linear or branched alkyl chain, with or without unsaturation, and having 8 to 10 carbon atoms, X is the xylose residue, and n represents the average degree of oligomerization and is between 1 and 3.

19. Method of formulation of cosmetic, pharmaceutical, phytopharmaceutical, or detergent products, for water treatment or soil decontamination, comprising dispersing vesicles or liposomes in the form of a water-in-oil emulsion, solvents or mixtures of solvents, microemulsion contains from 1 to 20 wt. % of vesicles or liposomes, said vesicles or liposomes comprising from 5 to 99 wt. % of at least one alkyl polyglycoside of formula RO(X)n in which R is a linear or branched alkyl chain, with or without unsaturation, and having 8 to 10 carbon atoms, X is the xylose residue, and n represents the average degree of oligomerization and is between 1 and 3.
